SEQUENCE LISTING (6 sequences)

SEQ ID NO: 1 - DNA, 20 bp, Artificial
  Primer
cataccatgcaacatgttca

SEQ ID NO: 2 - DNA, 20 bp, Artificial
  Primer
cagtttcacgttgaccttca

SEQ ID NO: 3 - DNA, 40 bp, Artificial
  Primer
aaaagcggccgcccaccatgagcccaaagtgcagtgctga

SEQ ID NO: 4 - DNA, 21 bp, Artificial
  Primer
ctgacaagttcaaagaattca

SEQ ID NO: 5 - DNA, 20 bp, Artificial
  Primer
cgctgtactttgagaggaga

SEQ ID NO: 6 - DNA, 30 bp, Artificial
  Primer
aaaaaagcttgtttagttcctgtcttcctt

(19) United States
(12) Patent Application Publication
(10) Pub. No.: US 2005/0215489 A1
(43) Pub. Date: Sep. 29, 2005

(54) Methods of treating diabetes using pde 11a inhibitors

(75) Inventor: Haren Vasavada, Hamden, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/504,695

(22) PCT Filed: Mar. 14, 2003
(86) PCT No.: PCT/US03/08132

§ 371 Date: Aug. 12, 2004

Related U.S. Application Data
(60) Provisional application No. 60/364,697, filed on Mar. 14, 2002.
(60) Provisional application No. 60/389,036, filed on Jun. 13, 2002.

(51) Int. Cl. A61K 31/704; A61K 31/455; A61K 31/4439; A61K 31/426; A61K 31/155; A61K 31/194

(52) U.S. Cl. 514/25; 514/356; 514/340; 514/369; 514/635; 514/571

(57) ABSTRACT

Methods of the invention relate to treatment of diabetes, particularly type 2 diabetes, and related disorders by administration of a PDE11A inhibitor. Such PDE11A inhibitors may be administered in conjunction with alpha-glucosidase inhibitors, insulin sensitizers, insulin secretagogues, hepatic glucose output lowering compounds, beta3 agonist or insulin. Such PDE11A inhibitors may also be administered in conjunction with body weight reducing agents. Further methods of the invention relate to stimulating insulin release from pancreatic cells, particularly in response to an elevation in blood glucose concentration, by administration of a PDE11A inhibitor.

### FIELD OF THE INVENTION

The invention relates to methods of treating diabetes and related disorders by administering a compound that inhibits PDE11A.

### BACKGROUND

Diabetes is characterized by impaired glucose metabolism manifesting itself among other things by an elevated blood glucose level in the diabetic patient. Underlying defects lead to a classification of diabetes into two major groups: type 1 and type 2. Type 1 diabetes, or insulin dependent diabetes mellitus (IDDM), arises when patients lack insulin-producing beta-cells in their pancreatic glands. Type 2 diabetes, or non-insulin dependent diabetes mellitus (NIDDM), occurs in patients with impaired beta-cell function and alterations in insulin action.

The current treatment for type 1 diabetic patients is the injection of insulin, while the majority of type 2 diabetic patients are treated with agents that stimulate beta-cell function or with agents that enhance the tissue sensitivity of the patients towards insulin. The drugs presently used to treat type 2 diabetes include alpha-glucosidase inhibitors, insulin sensitizers, insulin secretagogues, metformin and insulin.

Over time, more than one-third of all type 2 diabetic subjects lose their response to oral agents. Insulin treatment is instituted after diet, exercise, and oral medications have failed to adequately control blood glucose. The drawbacks of insulin treatment are the need for drug injection, the potential for hypoglycemia, and weight gain.

Another strategy for diabetes therapy is based on the cyclic adenosine monophosphate (cAMP) signaling mechanism and its effects on insulin secretion. Metabolism of glucose promotes the closure of ATP-dependent K⁺ channels, which leads to cell depolarization and subsequent opening of Ca⁺⁺ channels. This in turn results in the exocytosis of insulin granules. cAMP is a major regulator of glucose-stimulated insulin secretion. The effect of cAMP is, however, glucose-dependent, i.e., cAMP has little if any effects on insulin secretion at low glucose concentrations (Weinhaus, A., et al., Diabetes 47: 1426-1435 (1998)). The effects of cAMP on insulin secretion are thought to be mediated by a protein kinase A pathway.

Endogenous secretagogues use the cAMP system to regulate insulin secretion in a glucose-dependent fashion (Komatsu, M., et al., Diabetes 46: 1928-1938, (1997)). Examples of such endogenous secretagogues include pituitary adenylate cyclase activating peptide (PACAP), vasoactive intestinal polypeptide (VIP), and glucagon-like peptide-1 (GLP-1).

PACAP is a potent stimulator of glucose-dependent insulin secretion from pancreatic beta cells. Three different PACAP receptor types (R1, R2, and R3) have been described (Harmar, A. et al., Pharmacol. Reviews 50: 265-270 (1998)). The insulinotropic action of PACAP is mediated by the GTP binding protein Gs. Accumulation of intracellular cAMP in turn activates nonselective cation channels in beta cells increasing [Ca⁺⁺], and promoting the exocytosis of insulin-containing secretory granules.

Vasoactive intestinal peptide (VIP) is a 28 amino acid peptide that was first isolated from hog upper small intestine (Said and Mutt, Science 169: 1217-1218, 1970; U.S. Pat. No. 3,879,371). The biological effects of VIP are mediated by the activation of membrane-bound receptor proteins that are coupled to the intracellular cAMP signaling system.

GLP-1 is released from the intestinal L-cell after a meal and functions as an incretin hormone (i.e., it potentiates glucose-induced insulin release from the pancreatic beta cell). It is a 37-amino acid peptide that is differentially expressed by the glucagon gene, depending upon tissue type. The clinical data that support the beneficial effect of raising cAMP levels in β-cells have been collected with GLP-1. Infusions of GLP-1 in poorly controlled type 2 diabetics normalized their fasting blood glucose levels (Gutniak, M., et al., New Eng. J. Med. 326:1316-1322, (1992)) and with longer infusions improved the beta cell function to those of normal subjects (Rachman, J., et al., Diabetes 45: 1524-1530, (1996)). A recent report has shown that GLP-1 improves the ability of β-cells to respond to glucose in subjects with impaired glucose tolerance (Byrne M., et al., Diabetes 47: 1259-1265 (1998)).

The use of such endogenous secretagogues to treat type 2 diabetes also has some drawbacks. For instance, the peptidyl nature of these compounds requires that they be administered by injection. Additionally, the effects of the endogenous secretagogues are short-lived because of the short half-life of the peptides.

Because of the problems with current treatments, new therapies to treat type 2 diabetes are needed. In particular, new treatments to retain normal (glucose-dependent) insulin secretion are needed. Such new drugs should have the following characteristics: 1) dependency on glucose for promoting insulin secretion, i.e., compounds that stimulate insulin secretion only in the presence of elevated blood glucose and therefore low probability for hypoglycemia; 2) low primary and secondary failure rates; and 3) preservation of islet cell function. The present invention addresses these needs by focussing on regulation of the cAMP signaling system by inhibition of Phosphodiesterase 11A (PDE11A).

Phosphodiesterases (PDEs) are a family of cAMP and/or cGMP-hydrolyzing enzymes that cleave 3′,5′-cyclic nucleotide monophosphates to 5′-nucleotide monophosphates. PDEs are known to be involved in the regulation of the cAMP system. Specifically, PDE11A is a phosphodiesterase that hydrolyses cAMP and cGMP with Kₘ values of approximately 1-5 μM (Fawcett, et al., Proc Natl Acad Sci USA, 2000 Mar. 28; 97(7):3702-7 (2000); Hetman, et al., Proc Natl Acad Sci USA, 2000 Nov. 7; 97(23):12891-5 (2000); Yuasa, et al., Eur J Biochem, 2001 August; 268(16):4440-8 (2001)). At least four splice variants of PDE11A have been described that are identical in their C-terminal catalytic domains, but differ in the size of the N-terminal portion of the molecule. Yuasa, et al., Eur. J. Biochem. (2001), 268 (16), 4440-4448; Yuasa, et al., J. Bio. Chem. (2000), 275 (40), 31469-31479.

### SUMMARY OF THE INVENTION

The present invention relates to methods of treating diabetes, particularly type 2 diabetes, in a mammal by administering an effective amount of a PDE11A inhibitor. Other methods of the invention relate to treatment of other disorders related to diabetes, such as Syndrome X, impaired glucose tolerance and impaired fasting glucose, by administering a PDE11A inhibitor. The invention further relates to methods of stimulating insulin release from pancreatic cells in a mammal by administering an effective amount of a PDE11A inhibitor. This method of insulin release may be in response to an elevation of the concentration of glucose in the blood of a mammal. In methods of the invention, the PDE11A inhibitor may also be administered in conjunction with other diabetes therapies, such as alpha-glucosidase inhibitors (e.g., acarbose), insulin sensitizers (e.g., thiazolidinediones), compounds that reduce hepatic glucose output (e.g. metformin), insulin secretagogues (e.g., sulfonylureas), beta3-agonists, and insulin. Furthermore, the PDE11A inhibitor may be administered in conjunction with one or more weight reduction agents, such as Xenical, Meridia, a beta3-agonist or a CB-1 antagonist. Finally, in another embodiment, methods of the invention provide for the administration of a PDE11A inhibitor in combination with an HMG-CoA reductase inhibitor, nicotinic acid, a bile acid sequestrant, a fibric acid derivative, or an antihypertensive drug.

In other methods of the invention, a PDE11A inhibitor may be administered for the treatment of dementia or a urogenital tract disorder. Urogenital tract disorders include, but are not limited to, incontinence, stress incontinence, benign prostatic hyperplasia, erectile dysfunction, female sexual dysfunction, and hypertrophy of prostate. In other methods of the invention, a PDE11A inhibitor may be administered for the treatment of a cardiovascular disorder, such as hypertension, ischemic heart disease, myocardial infarction, stable and unstable angina, peripheral occlusive disease, and ischemic stroke.

The present invention therefore provides methods for the treatment of diabetes by inhibition of PDE11A through the administration of a PDE11A inhibitor. These and other aspects of the invention will be more apparent from the following drawings, description and claims.

### BRIEF DESCRIPTION OF THE DRAWINGS

FIGS. 1A-1C show the expression of PDE11A in islet cells.

FIG. 1A shows the PCR product generated from islet cDNA as template using the F2/R1 primer combination.

FIG. 1B shows the PCR product generated from islet cDNA using the For3/R2 primer combination.

FIG. 1C shows the PCR product generated from islet cDNA using the F4/Rev2 primer combination.

In the figures, arrows indicate PCR products with their predicted sizes. Lane identities are as follows: 1 Kb=1 Kb DNA standard markers, islet=islet cDNA template, −DNA=minus DNA control, PDE11A=positive control using plasmid containing PDE11A as template, Neg=negative control using plasmid containing an unrelated gene as template.

### DETAILED DESCRIPTION OF THE INVENTION

Methods of the invention provide for the treatment of diabetes and related disorders, particularly type 2 diabetes, and/or stimulation of insulin release from pancreatic cells, by the administration of a PDE11A inhibitor. Such methods provide for treatment of any condition in which glucose is elevated in the fasting or post-prandial state, by administration of a PDE11A inhibitor. PDE11A has been identified in islets of Langerhans. PDE11A hydrolyses cAMP to AMP and thereby decreases intracellular concentrations of cAMP. By inhibiting PDE11A activity, intracellular levels of cAMP are increased thereby increasing the release of insulin-containing secretory granules and therefore increasing insulin secretion. As shown herein, compounds that inhibit activity of PDE11A also stimulate insulin secretion in an islet assay. Also as described herein, a PDE11A inhibitor may be administered for the treatment of dementia, a cardiovascular disease or a urogenital tract disorder.

Methods of Treatment

Methods of the invention may be used to treat diseases, such as diabetes, including both Type 1 and Type 2 diabetes. Such methods may also delay the onset of diabetes and diabetic complications. Other diseases and conditions that may be treated or prevented using methods of the invention include: Maturity-Onset Diabetes of the Young (MODY) (Herman, et al., Diabetes 43:40 (1994)), Latent Autoimmune Diabetes Adult (LADA) (Zimmet, et al., Diabetes Med. 11:299 (1994)), impaired glucose tolerance (IGT) (Expert Committee on Classification of Diabetes Mellitus, Diabetes Care 22 (Supp. 1) S5 (1999)), impaired fasting glucose (IFG) (Charles, et al., Diabetes 40:796 (1991)), gestational diabetes (Metzger, Diabetes, 40:197 (1991), and metabolic syndrome X.

Methods of the invention may also be used to treat secondary causes of diabetes (Expert Committee on Classification of Diabetes Mellitus, Diabetes Care 22 (Supp. 1), S5 (1999)). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

cAMP-mediated release of insulin is also dependent on the presence of stimulatory glucose concentrations. A method of the invention further relates to stimulating insulin release from islet cells by the administration of a PDE11A inhibitor. Glucose-dependent stimulation of insulin secretion with non-peptide compounds therefore lowers blood glucose concentrations without causing hypoglycemia.

The methods of the present invention may be used alone or in combination with additional therapies and/or compounds known to those skilled in the art in the treatment of diabetes and related disorders. Alternatively, a PDE11A inhibitor may be used partially or completely, in combination therapy.

A PDE11A inhibitor may also be administered in combination with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, and insulin. Such therapies may be administered prior to, concurrently with or following administration of the PDE11A inhibitor. Insulin includes both long and short acting forms and formulations of insulin. PPAR agonist may include agonists of any of the PPAR subunits or combinations thereof. For example, PPAR agonist may include agonists of PPAR-α, PPAR-γ, PPAR-δ or any combination of two or three of the subunits of PPAR. PPAR agonists include, for example, rosiglitazone and pioglitazone. Sulfonylurea drugs include, for example, glyburide, glimepiride, chlorpropamide, and glipizide. α-glucosidase inhibitors that may be useful in treating diabetes when administered with a PDE11A inhibitor include acarbose, miglitol and voglibose. Insulin sensitizers that may be useful in treating diabetes when administered with a PDE11A inhibitor include thiazolidinediones and non-thiazolidinediones. Hepatic glucose output lowering compounds that may be useful in treating diabetes when administered with a PDE11A inhibitor include metformin, such as Glucophage and Glucophage XR. Insulin secretagogues that may be useful in treating diabetes when administered with a PDE11A inhibitor include sulfonylurea and non-sulfonylurea drugs: GLP-1, GIP, PAC/VPAC receptor agonists, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin. In one embodiment of the invention, a PDE11A inhibitor is used in combination with insulin secretagogues to increase the sensitivity of pancreatic beta cells to the insulin secretagogue.

A PDE11A inhibitor may be used in combination with anti-obesity drugs. Anti-obesity drugs include β-3 agonists, CB-1 antagonists, appetite suppressants, such as, for example, sibutramine (Meridia), and lipase inhibitors, such as, for example, orlistat (Xenical).

A PDE11A inhibitor may also be used in combination with drugs commonly used to treat lipid disorders in diabetic patients. Such drugs include, but are not limited to, HMG-CoA reductase inhibitors, nicotinic acid, bile acid sequestrants, and fibric acid derivatives. Methods of the invention may also be used in combination with anti-hypertensive drugs, such as, for example, β-blockers and ACE inhibitors.

Such co-therapies may be administered in any combination of two or more drugs (e.g., a PDE11A inhibitor in combination with an insulin sensitizer and an anti-obesity drug). Such co-therapies may be administered in the form of pharmaceutical compositions, as described above.

Other methods of the invention relate to administration of a PDE11A inhibitor for the treatment of dementia. Shimamoto, et al., Mechanisms of Aging Development (1976), 5 (4): 241-250; Nicholson, et al., Trends Pharmacological Sciences (1991), 12 (1): 19-27.

Still further methods of the invention relate to treatment of urogenital tract disorders by the administration of a PDE11A inhibitor. Such urogenital tract disorders include, but are not limited to, incontinence, stress incontinence, benign prostatic hyperplasia, erectile dysfunction, female sexual dysfunction (including female sexual arousal disorder), and hypertrophy of prostate. Ballard, et al., J Urology 159 (6): 2164-2171 (1998); EP 1211313A2 (for effects of PDE 11A on spermatogenesis).

Other methods of the invention relate to administration of a PDEl lA inhibitor to treat cardiovascular disorders, such as hypertension, ischemic heart disease, myocardial infarction, stable and unstable angina, peripheral occlusive disease, and ischemic stroke. The PDE11 family comprises enzymes which are responsible for the degradation of cAMP and cGMP in various tissues (Fawcett, et.al., PNAS (2000) 97, 3702-3707). Furthermore, expression of PDE11 can be detected in the heart (Yuasa, et.al., Eur. J. Biochem. (2001) 268, 4440-4448). Cyclic GMP (cGMP) and cyclic AMP (cAMP) are important second messengers which are involved in the regulation of vascular smooth muscle tone. The activation of soluble and membrane bound guanylate cyclases leads to increased intracellular cGMP levels and induces vasodilation. The stimulation of various G protein-coupled receptors (GPCRs) which are expressed in vascular smooth muscle cells (e.g. adrenomedullin and CGRP receptors) induces the activation of adenylate cyclases, generation of intracellular cAMP, and vasodilation. 3′,5′-cyclic nucleotide phosphodiesterases (PDEs) catalyze the hydrolysis of 3′,5′-cyclic nucleotides to their respective nucleoside 5′-monophosphates. For all of the reasons given above, PDE11A likely plays a role in the cardiovascular system.

Pharmaceutical Compositions

A PDE11A inhibitor for use in methods of the invention may be administered as compound per se. Alternatively, a PDE11A inhibitor may be administered with an acceptable carrier in the form of a pharmaceutical composition. The pharmaceutically acceptable carrier must be compatible with the other ingredients of the composition and must not be intolerably deleterious to the recipient. The carrier can be a solid or a liquid, or both, and preferably is formulated with the compound as a unit-dose composition, for example, a tablet, which can contain from about 0.05% to about 95% by weight of the active compound(s) based on a total weight of the dosage form. Other pharmacologically active substances can also be present, including other compounds useful in the treatment of a diabetic condition.

A PDE11A inhibitor for use in methods of the present invention may be administered by any suitable route, preferably in the form of a pharmaceutical composition adapted to such a route, and in a therapeutically effective dose for the treatment intended. The PDE11A inhibitor may, for example, be administered orally, sublingually, nasally, pulmonarily, mucosally, parenterally, intravascularly, intraperitoneally, subcutaneously, intramuscularly or topically. Unit dose formulations, particularly orally administrable unit dose formulations such as tablets or capsules, generally contain, for example, from about 0.001 to about 500 mg, preferably from about 0.005 mg to about 100 mg, and more preferably from about 0.01 to about 50 mg, of the active ingredient. In the case of pharmaceutically acceptable salts, the weights indicated above for the active ingredient refer to the weight of the pharmaceutically active ion derived from the salt.

For oral administration, the pharmaceutical composition may be in the form of, for example, a tablet, a capsule, a suspension, an emulsion, a paste, a solution, a syrup or other liquid form. The pharmaceutical composition is preferably made in the form of a dosage unit containing a particular amount of the active ingredient. If administered by mouth, the compounds may be admixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration.

Oral delivery of a PDE11A inhibitor can include formulations well known in the art to provide immediate delivery or prolonged or sustained delivery of a drug to the gastrointestinal tract by any number of mechanisms. Immediate delivery formulations include, but are not limited to, oral solutions, oral suspensions, fast-dissolving tablets or capsules, sublingual tablets, disintegrating tablets and the like. Prolonged or sustained delivery formulations include, but are not limited to, pH sensitive release of the active ingredient from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which an active drug molecule is delivered to the site of action by manipulation of the dosage form. Thus, enteric-coated and enteric-coated controlled release formulations may be used in methods of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the inhibitor(s) and the carrier (which can constitute one or more accessory ingredients). In general, the compositions are prepared by uniformly and intimately admixing the inhibitor(s) with a liquid or finely divided solid carrier, or both, and then, if necessary, shaping the product. For example, a tablet can be prepared by compressing or molding a powder or granules of the inhibitors, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a PDE11A inhibitor in a flavored base, usually sucrose, and acacia or tragacanth, and pastilles comprising the inhibitors in an inert base such as gelatin and glycerin or sucrose and acacia.

Formulations for parenteral administration, for example, may be in the form of aqueous or non-aqueous isotonic sterile injection solutions or suspensions. These solutions and suspensions may be prepared from sterile powders or granules having one or more of the carriers or diluents mentioned for use in the formulations for oral administration. A PDE11A inhibitor may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Pharmaceutically acceptable carriers encompass all the foregoing and the like. The pharmaceutical compositions containing PDE11A inhibitors for use in methods of the invention can be prepared by any of the well-known techniques of pharmacy, such as admixing the components. The above considerations in regard to effective formulations and administration procedures are well known in the art and are described in standard textbooks.

Dosage levels of the PDE11A inhbitors for use in methods of this invention typically are from about 0.001 mg to about 10,000 mg daily, preferably from about 0.005 mg to about 1,000 mg daily. On the basis of mg/kg daily dose, either given in a single or divided doses, dosages typically range from about 0.001/75 mg/kg to about 10,000/75 mg/kg, preferably from about 0.005/75 mg/kg to about 1,000/75 mg/kg.

The total daily dose of each inhibitor can be administered to the patient in a single dose, or in multiple subdoses. Typically, subdoses can be administered two to six times per day, preferably two to four times per day, and even more preferably two to three times per day. Doses can be in immediate release form or sustained release form sufficiently effective to obtain the desired control over the diabetic condition.

The dosage regimen to prevent, treat, give relief from, or ameliorate a diabetic condition or disorder, or to otherwise protect against or treat a diabetic condition with the combinations and compositions of the present invention is selected in accordance with a variety of factors. These factors include, but are not limited to, the type, age, weight, sex, diet, and medical condition of the subject, the severity of the disease, the route of administration, pharmacological considerations such as the activity, efficacy, pharmacokinetics and toxicology profiles of the particular inhibitors employed, whether a drug delivery system is utilized, and whether the inhibitors are administered with other active ingredients. Thus, the dosage regimen actually employed may vary widely and therefore deviate from the preferred dosage regimen set forth above.

Pharmaceutically-acceptable salts of the compounds useful as PDE11A inhibitors in methods of the present invention include salts commonly used to form alkali metal salts or form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, heterocyclic, carboxylic and sulfonic classes of organic acids. Examples of organic and sulfonic classes of organic acids includes, but are not limited to, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicyclic, galactaric and galacturonic acid and combinations thereof.

A PDE11A inhibitor for use in methods of the invention may also be administered as the pharmaceutically acceptable salt, protected acid, conjugate acid, tautomer, prodrug or stereoisomer of a compound found to inhibit the activity of PDE11A. Tautomers include, for example, hydroxy tautomers. Protected acids include, but are not limited to, protected acids such as esters, hydroxyamino derivatives, amides and sulfonamides. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995) which is hereby incorporated by reference). Commonly used prodrugs are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996), which is hereby incorporated by reference).

Besides being useful for human treatment, administration of a PDE11A inhibitor may also be useful for veterinary treatments of companion animals (e.g., horses, dogs, cats, etc.), exotic animals and farm animals. Even though the invention is described in terms of human biology, it is understood by those of ordinary skill in the art that the present invention is applicable to other mammals as well.

Expression Profiling

The expression of PDE11A in pancreatic islets was verified by PCR. PCR was performed using template DNA from an islet cDNA library with the following primer combinations that recognize different regions throughout the PDE11A gene:
F2 (5′-CATACCATGCAACATGTTCA-3′) plus R1 (5′-CAGTTTCACGTTGACCTTCA-3′), which would generate a predicted product of 928 basepairs.
For3 (5′-AAAAGCGGCCGCCCACCATGAGCCCAAAGTGCAGTGCTGA-3′; Note that this primer includes additional sequence for cloning purposes) plus R2 (5′-CTGACAAGTTCAAAGAATTCA-3′), which would generate a predicted product of 1060 basepairs.
F4 (5′-CGCTGTACTTTGAGAGGAGA-3′) plus Rev2

(5′-AAAAAAGCTTGTTTAGTTCCTGTCTTCCTT-3′; Note that this primer includes additional sequence for cloning purposes), which would generate a predicted product of 474 basepairs.

50 μl PCR reactions were assembled as follows:

| | |
|---|---|
| 5 μl | 10x Amplification Buffer (supplied with polymerase) |
| 5 μl | 10x PCR Enhancer (supplied with polymerase) |
| 1 μl | 50 mM MgSO₄ |
| 8 μl | 1.25 mM each dATP, dCTP, dGTP, dTTP |
| 1 μl | 100 μM Forward Primer |
| 1 μl | 100 μM Reverse Primer |
| 1 μl | Islet cDNA |
| 27 μl | H₂O |
| 1 μl | PLATINUM Pfx DNA Polymerase (Life Technologies) |

The reactions were cycled in a Perkin Elmer GeneAmp PCR System 9600 Thermocycler using the following parameters:
95° C. 2 min/35×(95° C. 30 sec/55° C. 30 sec/72° C. 2 min)/72° C. 10 min
1 μl Taq DNA Polymerase (Perkin Elmer) was added to the reactions for an additional 10 min at 72° C., and then the reactions were cooled to 4° C.

The reactions were loaded on 1% agarose TBE gels and electrophoresed at 150 V for 20 min. The PCR products were visualized by UV illumination after Ethidium Bromide staining.

The PCR product of the F2/R1 reaction was chosen for further characterization by DNA sequencing, as this product corresponded to the portion of the PDE11A gene encoding the catalytic domain. Briefly, the PCR band was excised from the gel and purified using Qiagen Gel Extraction Kit following the protocol supplied with the kit. Purified PCR product was inserted into the pcDNA3.1/V5/His-TOPO vector followed by transformation of TOP10 competent bacterial cells using the Eukaryotic TOPO TA Cloning Kit (Invitrogen), as described by the manufacturer. Five colonies were picked into 2 ml LB broth with 100 ug/ml carbenicillin and grown at 37° C. overnight. Miniprep DNA was prepared from the overnight cultures the presence of PCR product insert was verified by restriction enzyme analysis. The insert was positively identified as PDE11A by DNA sequencing.

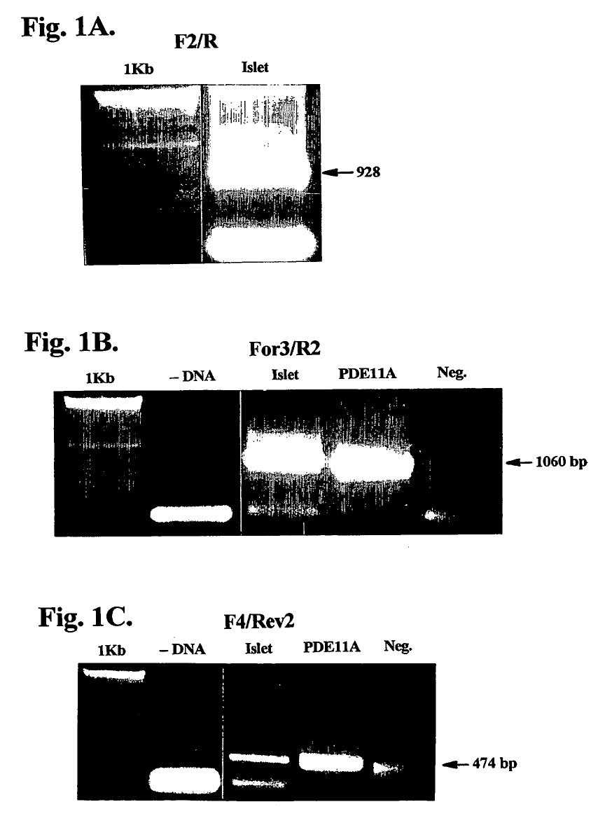

FIGS. 1A-1C show the PDE11A PCR products generated using the primer combinations described above. As shown in the figures, PDE11A is found in islet cells. Expression of PDE11A in islet cells indicates that PDE11A may have a role in regulating insulin release/blood glucose concentrations.

PDE11A Inhibition Assay

Compound is added to human recombinant enzyme in assay buffer to a 96-well whitewall/clear bottom isoplate (Wallac). The reaction is initiated by the addition of 3H-cAMP (Amersham) or 3H-cGMP (Amersham). After 45 minutes at room temperature, the reaction is stopped by the addition of SPA yttrium silicate beads (Amersham). After 30 minutes, the plate is read in the Microbeta (Wallac) for 30 seconds in the SPA mode. Data is expressed as a percentage of control. With PDE2, PDE3A, PDE4B, and PDE11A, 3H-cAMP was used as a substrate. With PDE5, 3H-cGMP was used as a substrate.

Compounds were identified that inhibited the activity of PDE11A with an IC₅₀ value of 1 μM or less. The compounds include the following:

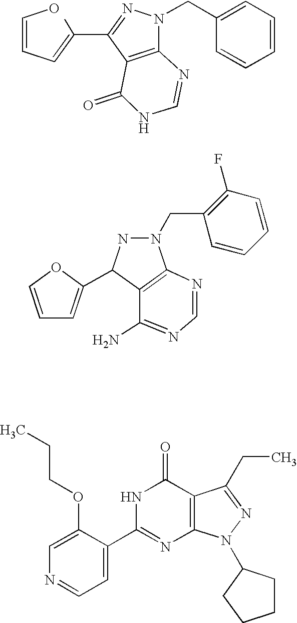

These same compounds were run in inhibition assays for PDE2, PDE3A, PDE4B and PDE5. In these assays, the compounds were found to have IC₅₀ values that were 10-fold greater than the IC₅₀ value for PDE11A. The compounds are therefore selective for PDE11A.

Compounds such as these may be administered in methods of the invention. Additionally, their stereoisomers, pharmaceutically-acceptable salts, tautomers, protected acids and the conjugate acids, and/or prodrugs may be administered in methods of the invention.

Islet Assay

Pancreatic islet isolation: Lean rats (Sprague-Dawley, male, 200-250 g) are anesthetized with nembutal (60 mg/kg, i.p.) and the abdomen opened to expose the liver and pancreas. The pancreas is distended by injection of Hank's solution into the bile duct, and then the pancreas is excised and minced with scissors while in Hank's solution. After rinsing the tissue with buffer, the pancreas is digested for ten minutes with collagenase, rinsed, and the islets separated from debris on a Ficoll gradient. The isolated islet fraction is rinsed with buffer, and the islets hand-picked under a microscope. The islets are pre-incubated in 3 mM glucose for 30 minutes and then transferred to media containing the appropriate conditions and incubated for an additional 30 minutes. The media is then assayed for insulin content using an ELISA kit (Alpco Diagnostics, Windham, N.H.).

Compounds identified as inhibitors of PDE11A in the PDE11A inhibition assay described above were tested in this islet assay. The compounds were also found to stimulate insulin release at least 1.5-fold over basal insulin release.

In Vivo Assay

Lean rats (Wistar, male, 250-300 g) are fasted overnight and divided into two groups: Vehicle and compound treatment (8 rats per group). Vehicle or compound is administrated via oral gavage (1.5 ml/rat). Two hours later, glucose solution (30%, 2 g/kg body weight) is injected intraperitoneally. Tail blood samples are collected at 0, 15, 30, and 60 min after glucose injection to measure blood glucose using Glucometer (Bayer Diagnostics, Mishawaka, Ind.).

Compounds identified in the PDE11A inhibition assay described above and tested in the islet assay described above are anticipated to have a blood glucose lowering effect when tested in this assay.

The invention may be embodied in other specific forms without departing from the spirit or essential characteristics thereof. The foregoing examples are included by way of illustration only. Accordingly, the scope of the invention is limited only by the scope of the appended claims.

1. A method of treating or preventing a disease or condition selected from the group consisting of diabetes, maturity-onset diabetes of the young (MODY), latent autoimmune diabetes adult (LADA), impaired glucose tolerance (IGT), impaired fasting glucose (IFG), gestational diabetes, and metabolic syndrome X, comprising administering to a mammal an effective amount of a PDE11A inhibitor.

2. The method of claim 1, wherein diabetes is type 2 diabetes.

3. The method of claim 1, further comprising administering a PPAR-agonist, an insulin sensitizer, a sulfonylurea, an insulin secretagogue, a hepatic glucose output lowering compound, an α-glucosidase inhibitor or insulin in combination with said PDE11A inhibitor.

4. The method of claim 3, wherein said PPAR-agonist is selected from rosiglitazone and pioglitazone.

5. The method of claim 3, wherein said sulfonylurea is selected from glibenclamide, glimepiride, chlorpropamide, and glipizide.

6. The method of claim 3, wherein said insulin secretagogue is selected from GLP-1, GIP, PAC/VPAC receptor agonists, secretin, nateglinide, meglitinide, repaglinide, glibenclamide, glimepiride, chlorpropamide, and glipizide.

7. The method of claim 3, wherein said α-glucosidase inhibitor is selected from acarbose, miglitol and voglibose.

8. The method of claim 3, wherein said hepatic glucose output lowering compound is metformin.

9. The method of claim 1, further comprising administering an HMG-CoA reductase inhibitor, nicotinic acid, a bile acid sequestrant, a fibric acid derivative, antihypertensive drug, or an anti-obesity drug in combination with said PDE11A inhibitor.

10. The method of claim 9, wherein said anti-obesity drug is selected from a β-3 agonist, a CB-1 antagonist, and a lipase inhibitor.

11. A method of treating or preventing secondary causes of diabetes selected from glucocorticoid excess, growth hormone excess, pheochromocytoma, and drug-induced diabetes, comprising administering to a mammal an effective amount of a PDE11A inhibitor.

12. A method of increasing the sensitivity of pancreatic beta cells to an insulin secretagogue, comprising administering to a mammal an effective amount of a PDE11A inhibitor.

13. The method of claim 12, wherein said insulin secretagogue is selected from GLP-1, GIP, PAC/VPAC receptor agonists, secretin, nateglinide, meglitinide, repaglinide, glibenclamide, glimepiride, chlorpropamide, and glipizide.

14. A method of treating or preventing dementia, comprising administering to a mammal an effective amount of a PDE11A inhibitor.

15. A method of treating or preventing a cardiovascular disorder selected from hypertension, ischemic heart disease, myocardial infarction, stable and unstable angina, peripheral occulusive disease and ischemic stroke, comprising administering to a mammal an effective amount of a PDE11A inhibitor.

16. A method of treating or preventing a urogenital tract disorder selected from incontinence, stress incontinence, benign prostatic hyperplasia, erectile dysfunction, female sexual dysfunction, and prostatic hypertrophy, comprising administering to a mammal an effective amount of a PDE11A inhibitor.

17. The method of claim 16, wherein said female sexual dysfunction is female sexual arousal disorder.
